(19) United States
(12) Patent Application Publication
(10) Pub. No.: US 2006/0018859 A1
(43) Pub. Date: Jan. 26, 2006

(54) Modified human serum albumin with reduced or eliminated affinity to chemical or biological contaminants at Cys 34

(76) Inventor: Daniel C. Carter, Huntsville, AL (US)

Correspondence Address: STITES & HARBISON PLLC, 1199 NORTH FAIRFAX STREET, SUITE 900, ALEXANDRIA, VA, 22314, US

(21) Appl. No.: 11/183,163

(22) Filed: Jul. 18, 2005

Related U.S. Application Data
(60) Provisional application No. 60/588,371, filed on Jul. 16, 2004.

(51) Int. Cl. A61K 38/38; A61K 8/64; C07H 21/04; C12P 21/04; C12N 15/09

(52) U.S. Cl. 424/70.14; 435/69.6; 435/320.1; 435/325; 530/363; 514/12; 536/23.5

(57) ABSTRACT

A modified serum albumin is provided which has been modified at position cysteine 34 of human serum albumin, or at equivalent locations in other mammalian serum albumins, with an amino acid which lacks a sulphydryl group and will thus be less reactive with biological or chemical molecules such as trace metals at this site. Particularly suitable amino acids to substitute for cysteine at position 34 include methionine and other amino acids which are less reactive than cysteine and which maintain the same folding and antigenicity of the native serum albumin. The modified albumin of the present invention is advantageous in that it is binding to trace metals and other contaminants is reduced or eliminated, and it can thus be used more safely and effectively than unmodified albumin with a reduced or eliminated likelihood of causing an allergic reaction in the human being treated with the albumin composition.

### CROSS-REFERENCE TO RELATED APPLICATIONS

This application claims the benefit of U.S. Provisional Application Ser. No. 60/588,371, filed Jul. 16, 2004, said application incorporated herein by reference.

### FIELD OF THE INVENTION

This invention relates in general to a modified serum albumin useful as a blood volume expander, and in particular to a recombinant or otherwise modified serum albumin that is mutated to have eliminated the single reactive sulphydryl at Cys34, thereby reducing a wide variety of undesired chemical reactions with small molecules, biologics and metal elements, such as gold, mercury, silver, nickel or copper so as to be safer and more effective in a variety of applications including use as a blood volume expander, for excipient and culture media applications, or as additives or substitutes in a wide range of products including medicines, cosmetics, dairy products and dietary supplements. These applications are improved over those prior forms which would normally utilize unmodified forms of serum albumin and thus potentially present problems caused by the presence of those sulphydryl complexes, such as toxicity and antigenicity. In particular, the invention relates to the provision of a modification in human serum albumin at the cysteine 34 position, or to other mammalian serum albumins at that position or the position equivalent to that position, so as to substantially reduce the tendency of serum albumin to bind to metals and other undesirable chemicals or biologics which would otherwise bind to the sulphydryl group of cysteine.

### BACKGROUND OF THE INVENTION

The serum albumins belong to a multigene family of proteins that includes alpha-fetoprotein and human group-specific component, also known as vitamin-D binding protein. The members of this multigene family are typically comprised of relatively large multi-domain proteins, and the serum albumins are the major soluble proteins of the circulatory system and contribute to many vital physiological processes. Serum albumin generally comprises about 50% of the total blood component by dry weight, and as such is responsible for roughly 80% of the maintenance of colloid osmotic blood pressure and is chiefly responsible for controlling the physiological pH of blood.

The albumins and their related blood proteins also play an extremely important role in the transport, distribution and metabolism of many endogenous and exogenous ligands in the human body, including a variety of chemically diverse molecules including fatty acids, amino acids, steroids, calcium, metals such as copper and zinc, and various pharmaceutical agents. The albumin family of molecules are generally thought to facilitate transfer many of these ligands across organ-circulatory interfaces such as the liver, intestines, kidneys and the brain, and studies have suggested the existence of an albumin cell surface receptor. See, e.g., Schnitzer et al., P.N.A.S. 85:6773 (1988). The albumins are thus intimately involved in a wide range of circulatory and metabolic functions.

Human serum albumin (HSA) is a protein of about 66,500 kD and is comprised of 585 amino acids including at least 17 disulphide bridges. As with many of the members of the albumin family, human serum albumin plays an extremely important role in human physiology and is located in virtually every human tissue and bodily secretion. As indicated above, HSA has an outstanding ability to bind and transport a wide spectrum of ligands throughout the circulatory system including the long-chain fatty acids which are otherwise insoluble in circulating plasma. The atomic structure and particular details regarding the binding affinities of albumin and the specific regions primarily responsible for those binding properties have been previously determined as set forth, e.g., in U.S. patent application Ser. No. 08/448,196, filed May 25, 1993, now U.S. Pat. No. 5,780,594 and U.S. patent application Ser. No. 08/984,176, filed Dec. 3, 1997, now U.S. Pat. No. 5,948,609, both of which are incorporated herein by reference.

In addition to human serum albumin, studies have been made on albumins in a variety of animal species, and it has been determined that over 60% of the amino acid sequences are conserved among the known albumin sequences of many mammals in addition to humans, including bovine, sheep, ovine, cat, dog, rat, and mice serum albumin. Sequences of the serum albumins from mammals have been disclosed in Peters, “All About Albumin”, Academic Press (1995), incorporated herein by reference. Furthermore, all members of the albumin multigene family for which sequences have been determined appear to have internal sequence homology (from two- to seven-fold), thus suggesting that the proteins evolved from a common ancestral protein, and reflecting the vital nature and function of this protein. See, e.g., Carter et al., Science 244:1195 (1989).

Because of the vital role played by albumins, there are literally thousands of applications for serum albumin and its related proteins covering a wide range of physiological conditions, and most often, native serum albumin has been used. However, unlike blood proteins such as hemoglobin, native serum albumins are non-functional as oxygen transport systems, and thus have not been useful in blood replacement systems requiring oxygen transport. More recently, an oxygen-transporting albumin-based blood replacement composition was developed which can be utilized as a blood volume expander, as has been disclosed in U.S. Pat. No. 5,948,609, incorporated herein by reference, and this composition further increases the importance and usefulness of serum albumin.

Additionally, in applications involving albumin, it has been known to utilize the human serum albumin sequence of the prototypical or major allotype of the human serum albumin sequence. See, e.g., Carter et al., Advances in Protein Chemistry, Vol. 45, 153-203 (1994) and Peters, “All About Albumin”, Academic Press (1995), both of said references incorporated herein by reference. However, such allotypes suffer because of undue binding of copper and nickel to the n-terminal peptide and other peptides, and at the physiological pH, some of the metals may be bound at extraordinarily high affinity. While this ability of albumin to bind to metals serves to protect the body from the potential damaging influences of the metals, especially copper, the nickel complex with albumin is known to elicit allergic reactions in some individuals, which occurs following ingestion of Ni(II) or exposure to nickel plated jewelry or other similar items. For example, an occupational asthma resulting from nickel binding is well recognized and has been traced to antibodies against Ni(II) specifically bound to the n-terminus of human serum albumin. See Carter et al. (1994), supra and Nieboer et al., Br. J. Ind. Med., Vol. 41:56-63 (1984).

In the normal course of recombinant production of albumin and other proteins, there is usually a given level of certain metals, including nickel and copper, and other chemical and biological agents, which are required as components of the culture media and used in albumin production, but which are undesirable in many application but still become bound to albumin. Consequently, significant levels of these undesirable chemical and biological agents, including trace metals such as nickel, copper and/or other metals are chelated by albumin during production, as evidenced during production by the green and yellow coloration of the recombinant human serum albumin. However, the presence of these contaminants, even in trace amounts, in the albumin produced via recombinant methods can lead to significant health problems as indicated above. One set of modifications developed to reduce metal binding to albumin are the forms of albumin as disclosed in U.S. Pat. No. 6,787,636, said patent incorporated herein by reference. However, it has not been known which other binding sites exist and if or how any such sites could be modified so as to further reduce the affinity of albumin to metals and other chemical or biological materials There is thus a significant need to develop safe and effective serum albumin products for use in many applications, particularly those which involve use in humans either internally or externally, which can reduce or eliminate the high affinity of albumin to certain chemicals, including metals such as copper and nickel and/or other metals, and undesirable biological agents, which can thus reduce the risk that a potential albumin-based or recombinantly produced product will elicit an allergic response to the bound antigen or metal in a human or animal who is being treated with an albumin composition.

### SUMMARY OF THE INVENTION

Accordingly, it is thus an object of the present invention to provide a novel serum albumin composition in which the single reactive sulphydryl at Cys34 is eliminated, thereby reducing a wide variety of undesired chemical reactions with small molecules, biologics and trace metals such as gold, mercury, silver, nickel and/or copper.

It is further an object of the present invention to provide a novel modified serum albumin having replaced the active sulphydryl of Cys34 with an amino acid that does not contain a sulphydryl group and will be less reactive than amino acids which contain this group, thereby reducing or eliminating the undesirable incorporation of contaminants which would otherwise bind at the sulphydryl group, and which can be utilized in a variety of applications including as a blood replacement product, culture media component, a cosmetic, a medicament, or a pharmaceutical additive.

It is still further an object of the present invention to provide a novel serum albumin product which has improved clarity and homogeneity so as to become more useful in scientific applications involving analytical spectroscopy.

It is even further an object of the present invention to provide a novel serum albumin-based blood replacement product which not only encompasses a variety of useful physiological functions including oxygen transport and expansion of blood volume, but which can be utilized safely in humans and animals and which has reduced or eliminated binding to biologics, small molecules and metals such as gold, mercury, silver nickel and/or copper that would otherwise bind the sulphydryl group at Cys34, and thus presents a greatly reduced risk of causing an allergic reaction or other harmful conditions.

It is even further an object of the present invention to provide a pharmaceutical or cosmetic composition having eliminated the single active sulphydryl found in all mammalian albumins to a variety of biologics, small molecules and trace metals such as nickel and/or copper which comprises the modified albumin of the invention along with a physiologically acceptable vehicle, carrier or excipient.

These and other objects are achieved by virtue of the present invention which provides a serum albumin which has been modified or mutated at the Cysteine 34 residue of human serum albumin (or its equivalent site on other mammalian serum albumins) in such a way as to disrupt the chemical reactivity of this binding site of the serum albumin at this position. In particular, this cysteine is preferably replaced by methionine or another amino acid which will be sufficient to hinder metal binding at this site while maintaining the appropriate albumin structure and thus reduce the affinity of the binding at that site and the binding region which includes Cysteine 34 to undesired chemicals or biological agents, in particular those which are reactive with sulphydryl groups, and trace metals. In this regard, many other amino acids which lack a sulphydryl group and which have less reactivity than cysteine can be substituted for the cysteine at position 34, including alanine, valine, serine, threonine, leucine, isoleucine, glycine, phenylalanine, tyrosine, aspartic acid, glutamic acid, glutamine, asparagine and lysine to provide a serum albumin with reduced affinity to metals, chemicals and other biological molecules. As a result of these modifications, which may be made either recombinantly or through other suitable physical or chemical means, the resulting serum albumin composition will have greatly reduced or totally eliminated chemical reactivity at the Cys34 position, and can thus be used safely and effectively in a variety of applications, including as a blood product, a cosmetic, a medicament, a pharmaceutical additive, or numerous other applications which presently employ albumin compositions.

These and other features of the present invention are set forth in, or will become obvious from, the detailed description of the preferred embodiments provided hereinbelow.

### DETAILED DESCRIPTION OF THE PREFERRED EMBODIMENTS

In accordance with the present invention, a modified serum albumin is provided which is modified at the Cysteine 34 position of human serum albumin (and the equivalent positions at other serum albumins) to replace the sulphydryl group of Cys34 with an amino acid that lacks a sulphydryl group so as to have reduced or eliminated affinity to such materials as certain chemicals, biological molecules and trace metals such as nickel and/or copper which would otherwise bind to the sulphydryl group. Accordingly, any mammalian serum albumin that has an equivalent residue to Cysteine 34 can be improved by the modification in accordance with the invention wherein the cysteine at that position is replaced by an amino acid lacking a sulphydryl group. In addition, it will also be the case that other peptides, fragments, or other subunits that contain the Cysteine 34 residue can also be improved by the present invention and will similarly have reduced binding to chemical contaminants and are thus contemplated as falling within the present invention. These peptides, fragments or other subunits of albumin can also be modified in the manner of the invention as set forth herein so as to reduce or eliminate unwanted binding of chemical or biological molecules which would be reactive to the sulphydryl group, including trace metals such as platinum, gold, mercury, silver, nickel or copper, in those applications wherein such reduction or elimination is desirable, and thus when reference is made to serum albumin, said reference includes reference to said peptides, fragments or other subunits when modified in like manner.

Cysteine 34 (or “Cys 34”) is a highly activated sulphydryl located on the surface of human serum albumin and other mammalian albumins. It reacts covalently with cysteine and many other activated small molecules and is responsible for destabilizing the storage life of albumin due to its inter molecular reaction to disulphides on other albumins. These reactions create dimers and higher molecular weight aggregates which are undesirable for medical applications. Additionally, Cys 34 is highly reactive to metals forming complexes with platinum, gold, silver and other trace metals, and with proteins and small molecules found in enriched fermentation media used to produce recombinant products. Many of the non-endogenous “post-translational” modifications at Cys 34 create undesirable and potentially dangerous impurities and contaminants (for injectable applications) which are difficult or impossible to remove from the final product. Accordingly, in accordance with the present invention, position 34 on human serum albumin, or its equivalent position on the other mammalian serum albumins as is well known in the art, is modified so that the amino acid at that position is replaced with one lacking a sulphydryl group so as to reduce or eliminate unwanted binding by metals and other undesirable impurities.

In accordance with the present invention, as indicated above, an improved serum albumin is provided wherein the Cysteine at position 34 of human serum albumin (or its equivalent in the albumins of other species) is replaced with an amino acid lacking a sulphydryl group which will thus be less reactive than the amino acid cysteine which includes the highly active sulphydryl group. In addition, it is preferred that the amino acid be selected which is one that does not affect the folding or antigenicity of human serum albumin, since there is mixed heterogeneity at Cys 34 naturally occurring in the circulation. In the particularly preferred from of the invention, the Cys34 residue is substituted by methionine which accomplishes this purpose by virtue of the fact that with methionine, the methyl group will covalently block the reactivity of the associated sulphydryl, thus resulting in (1) a dramatic reduction in the metal content of recombinantly produced albumin; (2) stability of the albumin and increased storage life; (3) elimination of the covalent reaction of undesirable foreign small molecules and proteins which could adversely affect the safety profile of plasma albumin; and (4) greatly improved homogeneity and color/clarity of the recombinant product. However, as set forth below, other amino acid substitutions are possible which will accomplish the same purpose and provide a serum albumin with reduced affinity to contaminants.

As indicated above, in accordance with the present invention, other amino acids which do not possess a sulphydryl group and which are thus less reactive than cysteine are suitable for use to substitute for the Cys 34 in accordance with the invention. In addition, it is also desirable that the amino acid residue chosen be one which otherwise does not have a major effect on the folding and affinity of the serum albumin, i.e., those amino acids which will have the least affect on the folding and antigenicity of the native serum albumin. In particular, those amino acids which can also be substituted at the Cys34 position in accordance with the invention will include alanine, valine, serine, threonine, leucine, isoleucine, glycine, phenylalanine, tyrosine, aspartic acid, glutamic acid, glutamine, asparagine and lysine, and these substitutions will result in an improved and safer or otherwise more desirable form of albumin for use in a variety of applications.

In the preferred mode of producing the modified albumins of the present invention, said albumins are produced via recombinant methods wherein the nucleic acids coding for the albumin proteins are genetically engineered so as to manufacture an albumin with the desired modifications at Cysteine 34 as set forth above. Accordingly, the present invention also contemplates the production, isolation and/or purification of nucleic acid sequences coding for the modified albumins of the present invention. However, numerous other methods which are conventionally employed to obtain modified proteins, such as physical, chemical or enzymatic methods, may also be suitable to produce the modified albumins of the invention as would be readily understood by one skilled in this art.

In accordance with the present invention, the modified serum albumins of the invention can thus be used in a variety of applications ranging from blood products and blood substitutes to cosmetics and other topical applications. In general, the modified albumins of the present invention, once prepared using any of the conventional methods set forth above which would be apparent to one of ordinary skill in this art, can be made into compositions that will be useful, e.g., as safe and effective blood products such as a blood volume expanders. As would also be recognized by one skilled in the art, the albumins of invention can be made into suitable blood replacement compositions in any of a variety of conventional methods well known in the art using physiologically acceptable fluids or other materials conventionally used in preparing other blood replacement products. Once prepared into physiologically compatible blood replacement solutions, the modified albumins of the present invention can be administered as needed to increase blood volume or, in the case of an albumin which has also been modified to transport oxygen, to enhance oxygen transport in the patient's circulatory system, for example, for patients who have suffered severe loss of blood, or during surgical operations. The modified albumins of the present invention may also be useable in a variety of other applications, such as those in the field of cosmetics or medical applications which currently employ albumin, either in the form of HSA or bovine serum albumin (BSA), or other similarly related compounds.

The modified albumins of the present invention may also be prepared into pharmaceutical compositions through introduction of the albumin in a physiologically acceptable vehicle, excipient or carrier. These pharmaceutical compositions would preferably be those compositions which would normally be prepared using non-modified albumin, and thus the preparation of these compositions in accordance with the present invention can be accomplished using conventional means already employed for such compositions, as would be understood by one skilled in this art.

In summary, the uses of the modified albumin of the present invention will be as expansive as those current uses of non-modified albumin and range from blood volume expanders, cosmetics, medicaments and pharmaceutical compositions as discussed above for possible use in shampoos, eye drop solutions, medicinal additives, and so on as would be recognized by one of ordinary skill in these arts. In addition, the large scale production of recombinant modified albumin will provide a safer and more tolerable albumin product and can potentially be employed in a variety of new applications including dairy products (e.g., nursing formulas, particularly for the many children that have been diagnosed as being allergic to bovine albumin of cows milk) and other dietary supplements (e.g., those where natural animal serum albumins would not be suitable). In all of these cases, the modified albumin of the invention with reduced affinity to trace metals will thus be far less likely to produce allergic reactions or other harmful conditions when compared to non-modified serum albumins which will have a significantly higher level of trace metals.

Finally, another important application contemplated by the present invention will be the utilization of the modified albumins of the invention in conjunction with the production of this protein through transgenic plants. The reason that the modified albumin of the invention will be particularly suitable to recombinant production in large quantities in transgenic plants is that such albumins will otherwise be very likely to pick up unwanted trace metals such as nickel or copper from the plants, and so the modification of the amino acids at cysteine 34 in accordance with the present invention will allow albumin to be produced in large quantities from transgenic plants without the potentially dangerous accumulation of metals and other chemical reactants that would otherwise occur.

As indicated above, the modified albumin of the present invention will result in a substantial and dramatic reduction in the undesirable contaminants such as metals and other biological or chemical reactants of recombinantly produced albumin; a more stable serum albumin which will have an increase in storage life and effectiveness; dramatically reduced or eliminated covalent reaction of undesirable foreign small molecules and proteins which could adversely affect the safety profile of plasma albumin; and a recombinant albumin product with improved homogeneity, color and clarity as compared to unmodified albumin whose color and clarity will be affected by binding to metals and other undesirable impurities.

It is thus submitted that the foregoing embodiments are only illustrative of the claimed invention, and alternative embodiments well known or obvious to one skilled in the art not specifically set forth above also fall within the scope of the claims.

What is claimed is:

1. An isolated human serum albumin having an amino acid lacking a reactive sulphydryl substituted for cysteine at position 34 of the amino acid sequence.

2. A pharmaceutical or cosmetic composition comprising the human serum albumin according to claim 1 and a physiologically acceptable vehicle, carrier or excipient.

3. The isolated human serum albumin according to claim 1 wherein the amino acid substituting for the cysteine at position 34 is selected from the group consisting of methionine, alanine, valine, serine, threonine, leucine, isoleucine, glycine, phenylalanine, tyrosine, aspartic acid, glutamic acid, glutamine, asparagine and lysine.

4. The isolated human serum albumin according to claim 1 wherein the substitution is achieved through recombinant means.

5. The isolated human serum albumin according to claim 1 that is produced using a transgenic plant.

6. The isolated human serum albumin according to claim 1 wherein the substitution is achieved through physical or chemical means.

7. An isolated nucleic acid molecule coding for the amino acid sequence of claim 1 or degenerates thereof.

8. An isolated human serum albumin having methionine substituted for cysteine at position 34 of the amino acid sequence.

9. A pharmaceutical or cosmetic composition comprising the human serum albumin according to claim 8 and a physiologically acceptable vehicle, carrier or excipient.

10. The isolated human serum albumin according to claim 8 wherein the substitution is achieved through recombinant means.

11. The isolated human serum albumin according to claim 8 that is produced using a transgenic plant.

12. The isolated human serum albumin according to claim 8 wherein the substitution is achieved through physical or chemical means.

13. An isolated nucleic acid molecule coding for the amino acid sequence of claim 8 or degenerates thereof.

14. An isolated mammalian serum albumin having an amino acid lacking a sulphydryl substituted for cysteine at the position equivalent to position 34 of the human serum albumin amino acid sequence.

15. A pharmaceutical or cosmetic composition comprising the serum albumin according to claim 14 and a physiologically acceptable vehicle, carrier or excipient.

16. The isolated mammalian serum albumin according to claim 14 wherein the substitution is achieved through recombinant means.

17. The isolated mammalian serum albumin according to claim 14 that is produced using a transgenic plant.

18. The isolated mammalian serum albumin according to claim 14 wherein the substitution is achieved through physical or chemical means.

19. An isolated nucleic acid molecule coding for the amino acid sequence of claim 18 or degenerates thereof.

20. An isolated mammalian serum albumin having a methionine substituted for cysteine at the position equivalent to position 34 of the human serum albumin amino acid sequence.

21. A method of reducing the affinity of human serum albumin to chemical or biological molecules which bind to a sulphydryl group comprising replacing cysteine at position 34 of the human serum albumin amino acid sequence with an amino acid lacking a sulphydryl.

22. The method of claim 21 wherein the amino acid substituting for the cysteine at position 34 is selected from the group consisting of methionine, alanine, valine, serine, threonine, leucine, isoleucine, glycine, phenylalanine, tyrosine, aspartic acid, glutamic acid, glutamine, asparagine and lysine.

23. The method of claim 21 wherein the chemical or biological molecule is a trace metal.

24. A method of reducing the affinity of a mammalian serum albumin to chemical or biological molecules which bind to a sulphydryl group comprising substituting an amino acid lacking a sulphydryl group for the cysteine at the position equivalent to position 34 of the human serum albumin amino acid sequence.

25. The method of claim 24 wherein the amino acid substituting for the cysteine at the position equivalent to position 34 of the human serum albumin is selected from the group consisting of methionine, alanine, valine, serine, threonine, leucine, isoleucine, glycine, phenylalanine, tyrosine, aspartic acid, glutamic acid, glutamine, asparagine and lysine.
